(19) United States
(12) Patent Application Publication
(10) Pub. No.: US 2013/0110198 A1
(43) Pub. Date: May 2, 2013

(54) Fixture and Removal of Hearing System External Coil

(71) Applicant: MED-EL Elektromedizinische Geraete GmbH, Innsbruck (AT)

(72) Inventor: Martin Stoffaneller, Innsbruck (AT)

(73) Assignee: MED-EL Elektromedizinische Geraete GmbH, Innsbruck (AT)

(21) Appl. No.: 13/661,324

(22) Filed: Oct. 26, 2012

Related U.S. Application Data
(60) Provisional application No. 61/552,230, filed on Oct. 27, 2011.

(51) Int. Cl. A61F 11/04

(52) U.S. Cl. 607/57

(57) ABSTRACT

A hair clamp for use with a partially implantable device is provided. The hair clamp includes a first magnet for magnetically interacting with both an implanted magnet of the partially implantable device, and a second magnet associated with an external component of the partially implantable device. A magnet support is coupled to the first magnet, the magnet support having a racket shape with a head portion and a handle portion extending therefrom, the first magnet coupled to the head portion.

### CROSS-REFERENCE TO RELATED APPLICATIONS

This application claims priority from U.S. provisional patent application Ser. No. 61/552,230 filed Oct. 27, 2011, entitled “Fixture and Removal of Hearing System External Coil,” which is hereby incorporated herein by reference in its entirety.

### TECHNICAL FIELD

The present invention relates to a system and methodology of fixing and/or removing an external coil of a device relative to a patient that includes a hair clamping technique.

### BACKGROUND ART

Cochlear implants and other inner ear prostheses are one option to help profoundly deaf or severely hearing impaired persons. Unlike conventional hearing aids that just apply an amplified and modified sound signal; a cochlear implant is based on direct electrical stimulation of the acoustic nerve. Typically, a cochlear implant stimulates neural structures in the inner ear electrically in such a way that hearing impressions most similar to normal hearing is obtained.

More particularly, a normal ear transmits sounds as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Some persons have partial or full loss of normal sensorineural hearing. Cochlear implant systems have been developed to overcome this by directly stimulating the user's cochlea 104. A typical cochlear prosthesis may include two parts: the speech processor 111 and the implanted stimulator 108. The speech processor 111 typically includes a microphone, a power supply (batteries) for the overall system and a processor that is used to perform signal processing of the acoustic signal to extract the stimulation parameters. The speech processor may be a behind-the-ear (BTE) device.

The stimulator 108 generates the stimulation patterns (based on the extracted audio information) that are sent through an electrode lead 109 to an implanted electrode array 110. Typically, this electrode array 110 includes multiple electrodes on its surface that provide selective stimulation of the cochlea 104. For example, each electrode of the cochlear implant is often stimulated with signals within an assigned frequency band based on the organization of the inner ear. The placement of each electrode within the cochlea is typically based on its assigned frequency band, with electrodes closer to the base of the cochlea generally corresponding to higher frequency bands.

The connection between the external speech processor 111 and the implanted stimulator 108 is usually established by means of a radio frequency (RF) inductive link. FIG. 1 shows the external, primary coil 107 of the inductive link. Note that via the inductive link both stimulation energy and stimulation information may be conveyed.

More particularly, as shown in FIG. 2, the inductive link typically includes a primary coil 205 positioned external to the skin 207, and a secondary coil 203 positioned underneath the skin 207. To keep the primary coil 205 in place, the two coils 203 and 205 are (preferable rigidly) connected to two permanent magnets 204 and 206, respectively, where the magnets are preferably positioned at the center of each coil. Positioning the magnets in this manner advantageously allows the magnets to align concentrically with each other, which ensures a good electromagnetically coupling. Furthermore, as shown in FIG. 3, the force F_{M} 311 of the magnet 206 results in a friction force F_{F}=F_{M}⊥*μₛ, where μₛ is the coefficient of static friction of the housing surrounding the primary magnet against the skin or hair of the patient. The friction force F_{F} 310 is summed with F_{MII} 312 (the component of F_{M} 311 that is parallel to the skin), to counter the force of gravity F_{G} 309.

Additionally, dynamic forces may add to the forces depicted in FIG. 3 (not drawn). These forces may have a parallel component (i.e., to the surface of the skin), which may add, or subtract, to the gravitational force, and must be taken into consideration. Such additional forces may be caused by, for example, linear acceleration (linear deceleration) or angular acceleration (angular deceleration) of the patients head.

Where the combination of the gravitational force F_{G} 309 and any dynamic force is stronger than the resultant force of friction F_{F} 310 and F_{MII} 312, the external primary coil/magnet component 206 may no longer remain properly positioned on the patient, and may fall off. Conventional solutions to this have been to increase the strength of magnets 204 and 206, or by adding supports that can hold the primary coil in place without magnetically attraction at all. For example the ear piece of glasses can serve as support for the primary coil housing. Other solutions for support include devices that use hair as an anchor to hold the primary coil with magnet and housing in place.

One known hair anchor approach includes a magnetic hair clip 401, as shown in FIG. 4. The magnetic clip 401 is made of a steel and is placed between the housing of magnet 206 (shown in FIG. 2) and the skin 207. The hair clip 401 is magnetically attracted to the housing of magnet 206, and with hair caught between the hair clip 401 and the housing of magnet 206, aids in supporting the housing of magnet 206. However, such a design adversely may affect both the magnetic field between the magnets 204 and 206, and the transmission rf-field (radiofrequency) characteristics of the coils 203 and 205.

Further disadvantages of the above-described prior art methodologies for maintaining the housing of magnet 206 properly positioned include that any additional parts needed may enlarge the housing of the primary coil and/or make it heavier. This may result in worse coaxial alignment of the two magnets, and consequently the coils, since the ratio of magnetic attraction force to weight gets worse. Additionally, the capability to easily remove the external primary coil/magnet component when desired may be adversely affected, and comfort and/or aesthetic appeal may be decreased.

### SUMMARY OF THE EMBODIMENTS

In accordance with a first embodiment of the invention, a hair clamp for use with a partially implantable hearing device is provided. The hair clamping includes a first magnet for magnetically interacting with both an implanted magnet of the partially implantable hearing device, and a second magnet associated with an external component of the partially implantable hearing device. A magnet support is coupled to the first magnet, the magnet support having a racket shape with a head portion and a handle portion extending therefrom, the first magnet coupled to the head portion.

In accordance with related embodiments of the invention, the handle portion of the hair clamp may extend along a longitudinal axis from the head portion, the handle portion having an elevated portion along the longitudinal axis. The elevated portion may be elevated to substantially the height of the external component when the magnet of the external component is juxtaposed the first magnet. A plurality of magnets may be coupled to the head portion. The shape of the head portion may be, for example, circular, rectangular, square, oval, elliptical and/or triangular. A coating of a material may be placed over the first magnet, the material providing increased friction relative to the first magnet. The handle portion may be removably attached to the head portion. The first magnet may be molded into the head portion.

In accordance with further related embodiments of the invention, the external component may include a first coil, and the implantable component includes a second coil. The first coil and the second coil used for transcutaneous transmission of at least one of a data signal and a power signal via electromagnetic coupling. The partially implantable hearing system may be a cochlear implant or a middle ear implant.

In accordance with another embodiment of the invention, a partially implantable hearing system includes a hair clamp having a first magnet. The first magnet is coupled to a magnet support. The hearing system further includes an external component of the partially implantable hearing device, the external component including a housing having a second magnet. An implantable component of the partially implantable hearing device includes a third magnet. When the second magnet is juxtaposed and aligned with the first magnet, the magnet support extends beyond the housing of the external component.

In accordance with related embodiments of the invention, the magnet support may have a racket shape with a head portion and a handle portion extending therefrom, the first magnet coupled to the head portion, the handle portion extending beyond the external component when the second magnet is juxtaposed and aligned with the first magnet. The head portion may have a shape that is one of circular, rectangular, square, oval, elliptical and triangular. The handle portion may extend along a longitudinal axis from the head portion, with the handle portion has an elevated portion along the longitudinal axis. The elevated portion may be elevated to substantially the height of the housing of the external component when the magnet of the external component is juxtaposed the first magnet. A plurality of magnets may be coupled to the magnet support.

In accordance with related embodiments of the invention, the external component may include a first coil, and the implantable component includes a second coil. The first coil and the second coil may be used for transcutaneous transmission of at least one of a data signal and a power signal via electromagnetic coupling. The partially implantable hearing system may be a cochlear implant or a middle ear implant.

In accordance with another embodiment of the invention, a method of positioning and removing an external component of a partially implantable hearing system relative to a user, respectively, is provided. The partially implantable hearing system includes a hair clamp that includes a first magnet held by a magnet support. The external component including a second magnet. The first magnet is positioned proximate a third magnet implanted in the user, such that hair clamping device is retained in a first position by magnetic forces between the first magnet and the third magnet. The second magnet is positioned proximate the first magnet, such that the external component is retained in a second position by magnetic forces between the second magnet and the first magnet, and the user's hair is caught between the hair clamp and the external component. When the second magnet is juxtaposed and aligned with the first magnet in the second position, an extended portion of the magnet support extends beyond the housing of the external component.

In accordance with related embodiments of the invention, the method may include holding down, by the user, on the extended portion of the magnet support. The external component is then removed from the second position, such that the external component is no longer retained in the second position by magnetic forces between the second magnet and the first magnet. Removing the external component may include sliding, by the user, the external component, until the magnetic fields of the second magnet is magnetically repelled away from the hair clamp. The magnet support may have a racket shape with a head portion and a handle portion extending therefrom, the first magnet coupled to the head portion.

In accordance with related embodiments of the invention, the external component may include a first coil, and the implantable component includes a second coil. The first coil and the second coil may be used for transcutaneous transmission of at least one of a data signal and a power signal via electromagnetic coupling. The partially implantable hearing system may be a cochlear implant or a middle ear implant.

### BRIEF DESCRIPTION OF THE DRAWINGS

The foregoing features of embodiments will be more readily understood by reference to the following detailed description, taken with reference to the accompanying drawings, in which:

FIG. 1 shows anatomical structures of a human ear and some components of a typical cochlear implant system;

FIG. 2 (Prior Art) shows an inductive link including primary and secondary coils;

FIG. 3 (Prior Art) shows forces acting on an external component;

FIG. 4 (Prior Art) shows a conventional magnetic hair clamp;

FIG. 5(a) shows a partially implantable hearing system including a hair clamp, in accordance with an embodiment of the invention; FIG. 5(b) shows the magnets of FIG. 5, in accordance with an embodiment of the invention; and FIG. 5(c) shows a hair clamp, in accordance with an embodiment of the invention;

FIG. 6 shows a hair clamp, in accordance with an embodiment of the invention;

FIG. 7 shows the pressing down of, or otherwise holding of, the extended portion (e.g., the handle portion) of the magnet support, in accordance with an embodiment of the invention;

FIG. 8 shows removal of the external component of the partially implantable hearing system, in accordance with an embodiment of the invention;

FIG. 9 shows a hair clamp that includes a handle portion with an elevated portion, in accordance with another embodiment of the invention;

FIG. 10 shows a hair clamp that includes a plurality of magnets, in accordance with an embodiment of the invention;

FIG. 11 shows a partially implantable hearing system that includes a hair clamp with a magnetic release, in accordance with an embodiment of the invention;

FIG. 12 shows a partially implantable hearing system that includes a hair clamp with a mechanical release, in accordance with an embodiment of the invention;

FIG. 13 shows a partially implantable hearing system that includes a hair clamp with an electromagnetic release, in accordance with an embodiment of the invention;

FIG. 14 shows a hair clamp and external component configuration that minimizes coil distance to the implanted coil (not shown), in accordance with an embodiment of the invention;

FIG. 15 shows use of two hair clamps, each with an associated magnet, in accordance with an embodiment of the invention;

FIG. 16 shows an ear clamp, in accordance with an embodiment of the invention;

FIG. 17 shows a hair clamp fixed to the skin of a user, in accordance with an embodiment of the invention; and

FIG. 18 shows two hair clamps, with one hair clamp fixed to the skin of the user, in accordance with an embodiment of the invention.

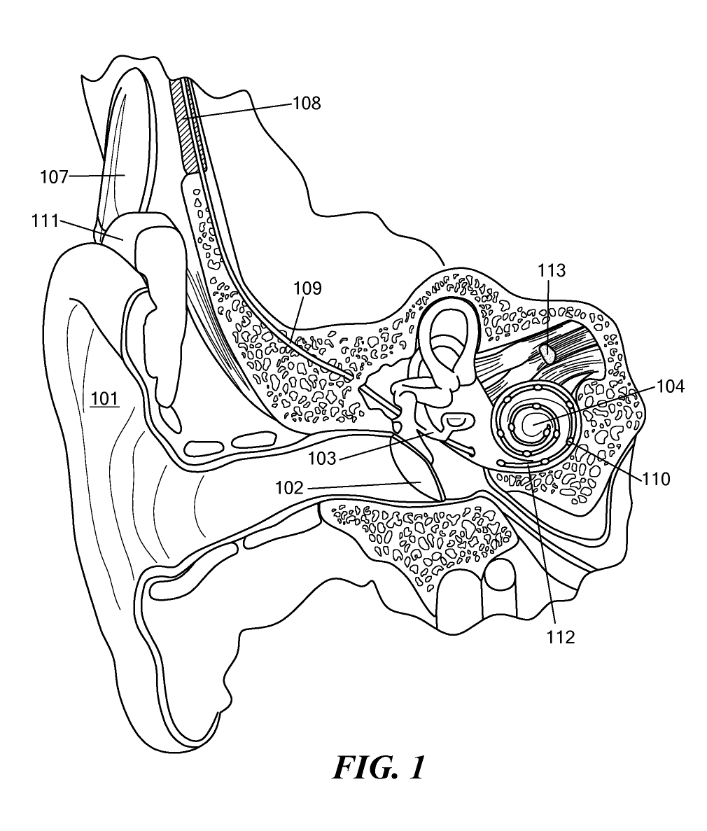

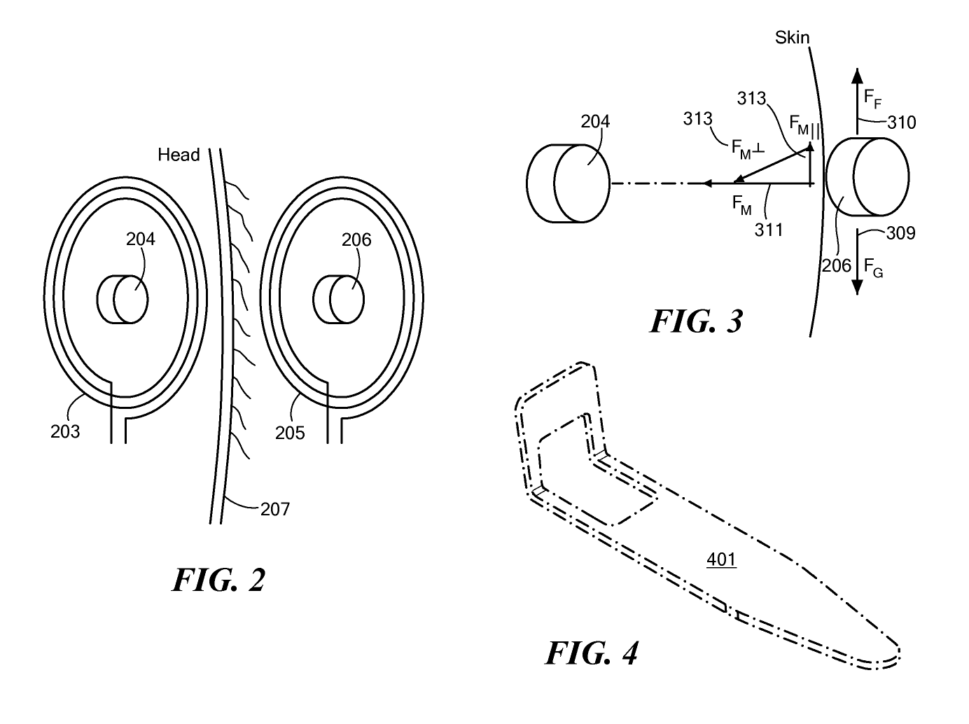

### DETAILED DESCRIPTION OF SPECIFIC EMBODIMENTS

In illustrative embodiments of the invention, a hair clamp for a partially implantable system such as a hearing system is provided. The hair clamp assists in retaining an external component of the partially implantable system properly positioned on a patient, and is advantageously shaped to allow for easy removal of the external component when desired. The partially implantable system may be associated with a cochlear implant or middle ear implant. Details are described below.

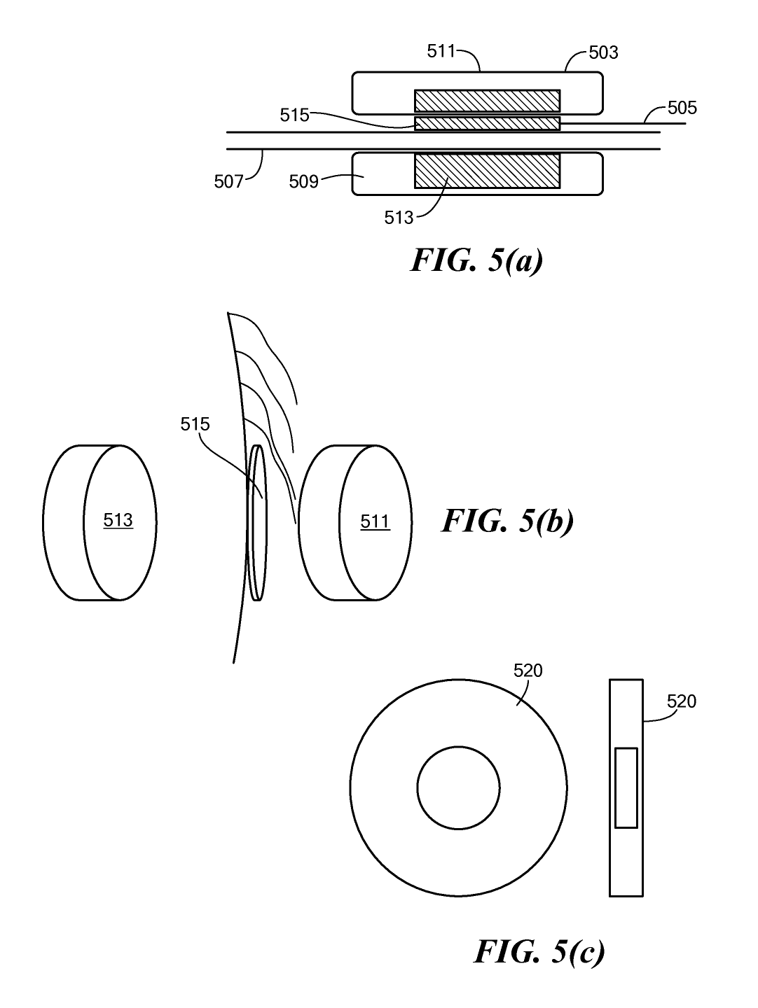

FIG. 5(a) shows a partially implantable hearing system, in accordance with an embodiment of the invention. The system includes: a hair clamp 505; an external component 503; and an implantable component 509 for implantation under the skin 507 of a user. Each of the hair clamp 505, the external component 503, and the implantable component 509 includes a magnet (first magnet 515, second magnet 511 and third magnet 513, respectively). The hair clamp 505 is configured for disposition between the external component 503 and the skin/implantable component 503, which are retained in proper position relative to the user by magnetic forces between associated magnets 515, 511 and 513 when juxtaposed.

The function of the hair clamp 505 is that any hair caught between first magnet 515 of the hair clamp 505 and second magnet 511 of the external component 503 advantageously adds friction against gravity and/or other dynamic forces, such that the external component 503 remains properly positioned on the patient, as shown in FIG. 5(b). If the first magnet 515 of the hair clamp 505 is designed/considered a slice of the second magnet 511 of the external component that is transferred out of the external component 503, no additional weight and volume is added. If realized as an additional slice, the magnetic force of the first magnet 515 increases the magnetic field of the second magnet 511 associated with the external component 503 and adds the additional effect of contributing against gravitational force by the clamping mechanism.

The partially implantable system may be, without limitation, a cochlear implant system, a middle ear implant system, a bone conduction implant system, a vestibular implant system, a retinal implant system, or any other implant system which comprises an external coil placed beneath a bundle of hairs on the skin of the body. For example, the external component 503 may include a first coil, and the implantable component 509, a second coil, the first coil and the second coil for transcutaneous transmission of at least one of a data signal and a power signal via electromagnetic coupling. The external component 503 may further include, and/or be operatively coupled to a speech processor. The speech processor may include, without limitation, the power supply (batteries) of the overall system and may be used to perform signal processing of the acoustic signal to extract stimulation parameters. The stimulation parameters may be provided, via the first coil and the second coil, to an implanted stimulator operatively coupled to the implantable component 509. The implanted stimulator then generates stimulation patterns and may mechanically conduct them to the middle ear (e.g., a middle ear implant), or the nervous tissue by means of an electrode array which usually is positioned in the scala tympani in the inner ear (e.g., a cochlear implant).

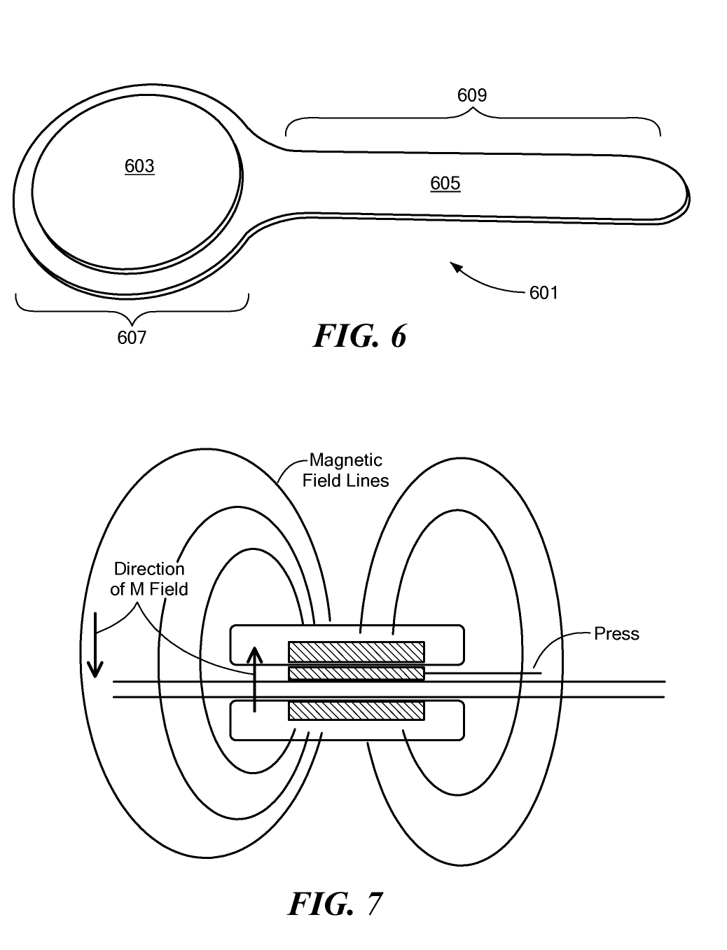

FIG. 6 shows a hair clamp 601 in more detail, in accordance with an embodiment of the invention. The hair clamp 601 includes a first magnet 603. The first magnet 603 is coupled to a magnet support 605 using, for example, various adhesives. Alternatively, the first magnet may be molded into place onto or within the magnet support 605. The magnet support 605 may be made from a variety of materials, including, without limitation, silicone.

A layer of material may be placed over the first magnet 603, with a higher coefficient of friction than first magnet 603, for increased friction, improved clamping capability, and/or protection against corrosion. The layer of material may be, without limitation, a silicon coating. In various embodiments, the coating may be adjustable, so as to provide a choice of friction coefficients for the patient. This may be accomplished, without limitation, by using a chemical liquid or more simply, by just exchanging different hair clamps. In this manner, the patient is able to adapt the friction to an amount that ensures, that for strong accelerations, the second magnet of the external component magnet slips before the patients hair or root of the hair is destructed or torn off. This safety option could be advantageous for patients with thin hair or weak roots of hair.

In illustrative embodiments of the invention, when the second magnet of the external component is juxtaposed/aligned with the first magnet of the hair clamp, the magnet support may extend beyond the housing of the external component (see, for example, FIG. 5). For example, in various embodiments the magnet support 605 may have, without limitation, a racket shape that includes a head portion 607 and a handle portion 609 extending therefrom, with the first magnet 603 attached to the head portion 607. The handle portion 609 extends beyond the external component when juxtaposed/aligned with the external component. The head portion may be, without limitation, circular, rectangular, square, oval, elliptical and/or triangular in shape. The handle portion 609 may be removably attached to the head portion 607. It is to be understood that the magnet support itself may be of any shape (e.g., circular, rectangular, square, oval, elliptical and/or triangular) and is not limited to a racket shape. For example, FIG. 5(c) shows top and cross-sectional views of a hair clamp 520 having a circular magnet support that may radially extend beyond the external component when juxtaposed.

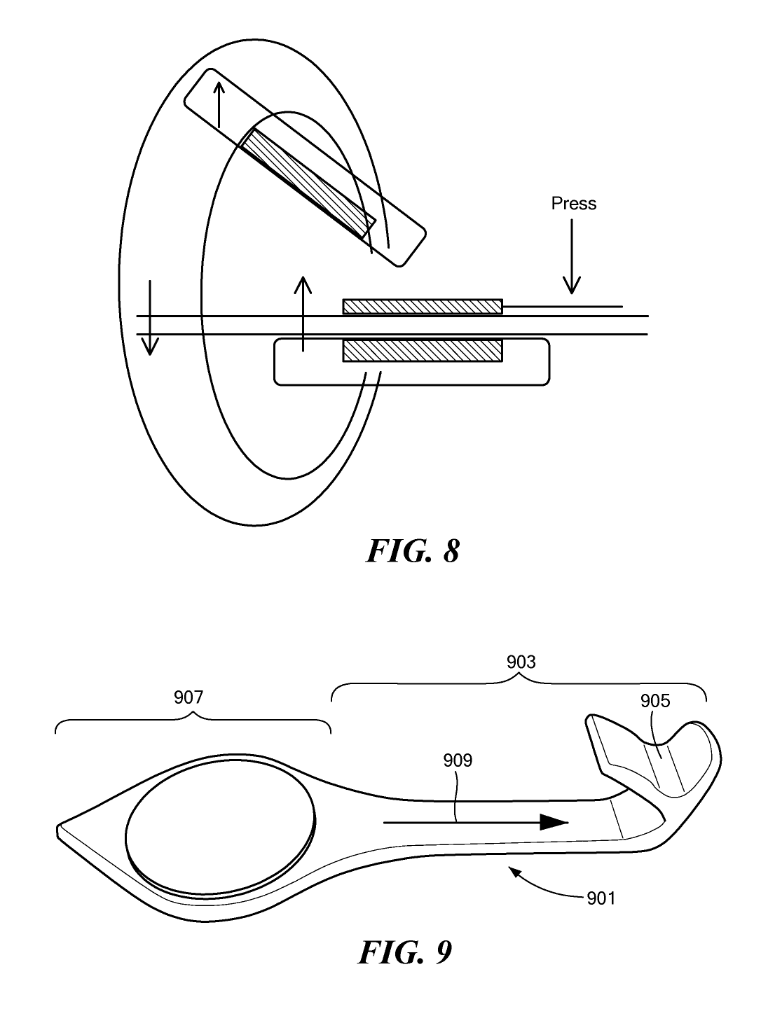

That the magnet support extends beyond the housing of the external component advantageously allows for easy removal of the external component from the hair clamp. Initially, when carrying the external device, the magnets of the external processor, hair clamp, and implantable component are aligned and are attracting each other. To remove the external component, the user may press down upon or otherwise holds the extended portion (e.g., the handle portion) of the magnet support to hold the hair clamp in place as shown in FIG. 7, in accordance with an embodiment of the invention. The user then slides, pushes or otherwise moves the external component into the repelling portion of the resulting magnetic field (i.e., the region of the magnetic field lines of the first and third magnet where the direction of the magnetic dipole field are opposite in direction), as shown in FIG. 8, in accordance with an embodiment of the invention. The external device is pushed away from the head/hair clamp, without pulling on any hair that was caught between the external device and hair clamp.

Additionally, enlarging the surface area of the magnet support may enlarge the area that contacts the user's skin, particularly if the thickness of the magnet support is very thin. This may advantageously aid certain patients in preventing tissue degradation due to the high pressure from the magnetic force between the internal and external components/hair clamp. However, care must be taken in that in various embodiments, the enlarged surface of the magnet support may not increase direct contact of the magnet support with the skin. In various embodiments, the release of pressure may be achieved by the use of weaker magnets resulting in weakened attracting force towards the implant since it does no longer have to compensate for the total gravitational force of the external component.

To remove the hair clamp that is left juxtaposed the implantable component, the patient may grasp the handle or head of the hair clamp, and slide the hair clamp in a direction parallel the skin and/or the plane spanned by the coil(s) to weaken the magnetic attraction. In embodiments without a hair clamp handle, a tiny cord attached to the hair clamp may be, without limitation, implemented. A hair clamp that is simply attracted to the external component (without being juxtaposed the implantable component) may be detached in a similar manner.

FIG. 9 shows a hair clamp, in accordance with another embodiment of the invention. The handle portion 903 of the hair clamp 901, extending along a longitudinal axis 909 from the head portion 907, includes an elevated portion 905 along the longitudinal axis 909. The elevated portion may be located, for example, towards the end of the handle portion 903 most distal to the head portion 907 of the hair clamp 901. The elevated portion 903 may assist the user in pressing down upon or otherwise holding the extended portion (e.g., the handle 903) of the hair clamp 901. The elevated portion 905 may be, without limitation, elevated to substantially the height of the external component when the magnet of the external component is juxtaposed the first magnet of the hair clamp.

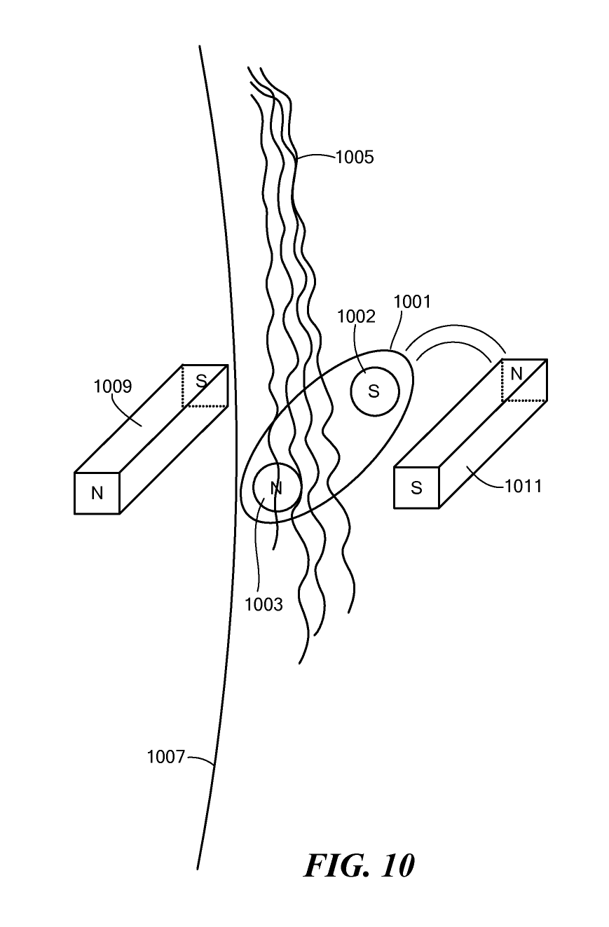

FIG. 10 shows a hair clamp that includes a plurality of magnets, in accordance with an embodiment of the invention. For example, the hair clamp 1001 may include magnets 1002 and 1003 with magnetic fields of these magnets perpendicular to the skin 1007. The hair of the patient may be clamped between the housing of the external component/second magnet 1011 and the portion of the magnet support of hair clamp 1001 between magnets 1002 and 1003 (which may be elevated relative to the magnet support).

Furthermore, the third magnet 1009 associated with the implantable component (and/or first and second magnets) may have a degree of freedom to rotate in at least one dimension. Such embodiments may be advantageous for implant compatability with strong external magnetic fields (e.g., in MRI scanners). A magnet design with such magnets (where at least the implanted one has a degree of freedom in at least one dimension) can be advantageous for implant compatibility with strong external magnetic fields (for e.g. in MRI scanners).

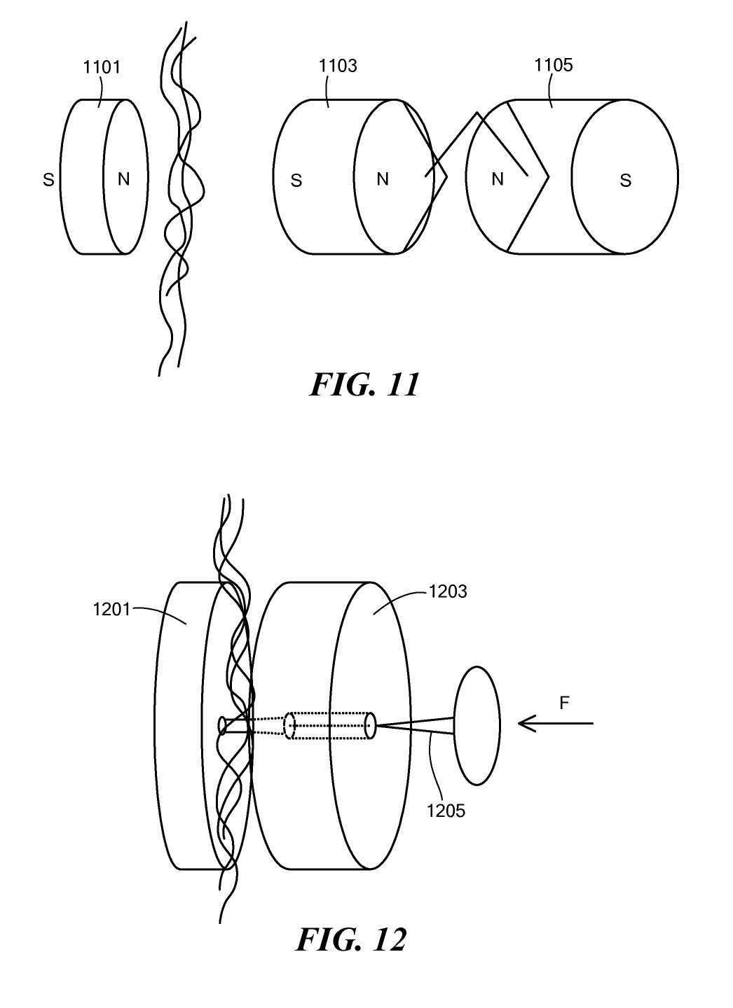

FIG. 11 shows a partially implantable hearing system that includes a hair clamp 1101 with a magnetic release, in accordance with an embodiment of the invention. More particularly, the system includes a first magnet 1101 of a hair clamp, a second magnet 1103 of an external component, a third magnet (not shown) of the implantable component, and furthermore, an additional fourth magnet 1105 to assist in releasing the external component from the first magnet 1101 when desired. The fourth magnet 1105, which may be stronger than the second magnet 1103 of the external component, may be placed adjacent the second magnet 1103 such that they are repelled against each other. Moreover, a resulting repulsive force is generated on the first magnet 1101 of the hair clamp. The second magnet 1103 and fourth magnet 1105 may have conforming truncated cone shape to prevent the magnets from repelling in a direction parallel to the skin.

FIG. 12 shows a partially implantable hearing system that includes a hair clamp 1201 with a mechanical release, in accordance with an embodiment of the invention. More particularly, the system includes a first magnet 1201 of a hair clamp and a second magnet 1103 of an external component. A pin, bolt or other shaped element, that may be spring loaded, can be guided by a hole through the external magnet 1203 and/or external component against the hair clamp and/or first magnet, to push and release the second magnet. The pin shape element could also include a locking mechanism for locking the hair clamp 1201 to the first magnet and/or hair clamp. For example, the hair clamp and/or first magnet may also include a hole to which the pin can enter, whereupon rotation of the pin locks the first magnet and hair clamp together.

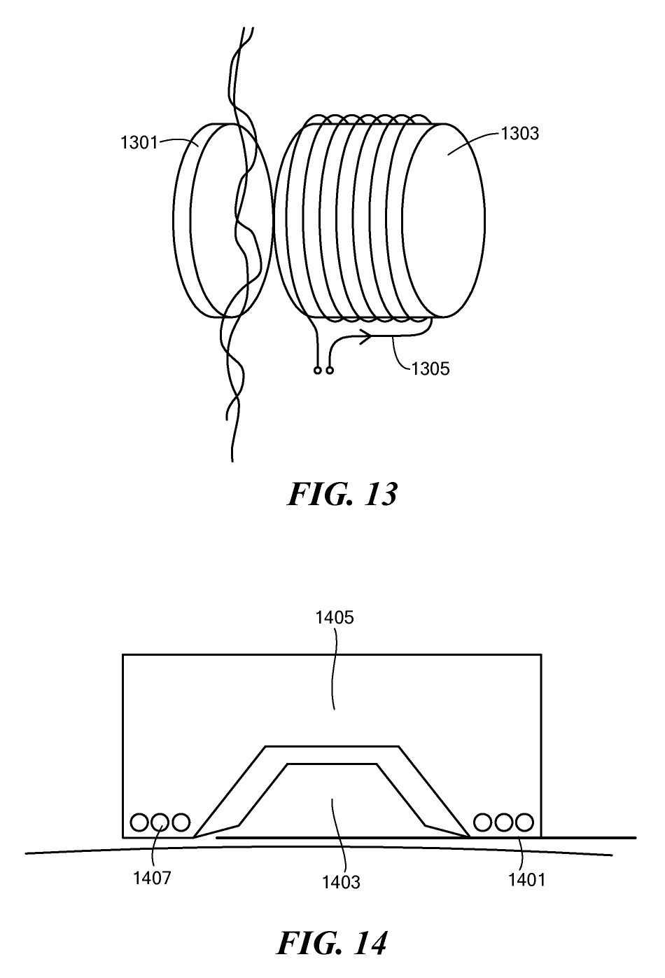

FIG. 13 shows a partially implantable hearing system that includes a hair clamp 1301 with an electromagnetic release, in accordance with an embodiment of the invention. A “release” coil 1305 associated with the external component 1303, upon receiving a current, may generate a magnetic field that releases the external component 1303 from hair clamp 1301. The current may be generated upon, without limitation, activating a switch on the external component 1303. Note that in alternative embodiments, the hair clamp 1301 may include the release coil and associated switch.

The hair clamp may be left on the patient even when the external component is removed, or the hair clamp may need additional support when used in combination with the external component. Thus, in some embodiments, it may be advantageous to further fix the hair clamp and/or associated magnet, for example, by skin glue or other adhesive or link, to establish a durable connection. In various embodiments the hair clamp may be glued or tied to the surrounding hair or other part of the head.

FIG. 14 shows a hair clamp 1401 and external component 1405 configuration that minimizes coil distance to the implanted coil (not shown), in accordance with an embodiment of the invention. The external component 1405 includes a cavity over which the first magnet 1403 fits, allowing the external coil 1401 to be positioned close to the skin.

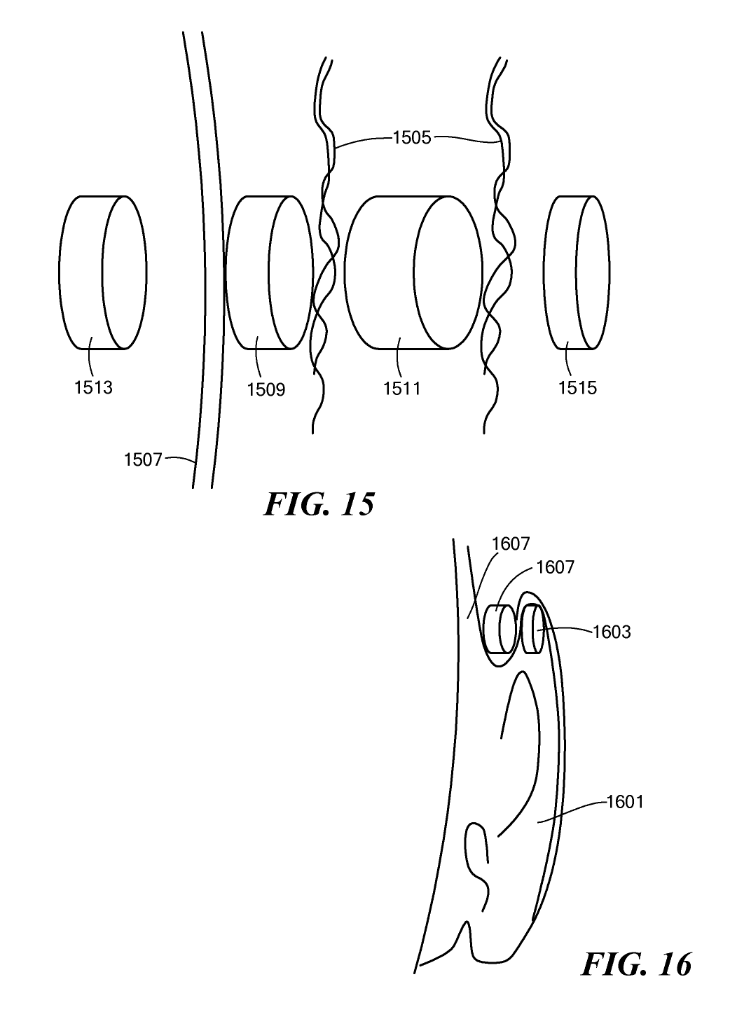

In accordance with another embodiment of the invention, FIG. 15 shows an embodiment that uses two hair clamps 1509 and 1515, each with an associated magnet. Hair clamp 1509 is disposed between the external component 1511 and the skin/implantable component 1513, which are retained in proper position relative to the user by magnetic forces between their associated magnets when juxtaposed. As described above, hair clamp 1509 may include, in addition to a magnet, a magnet support (not shown), that for example, is shaped like a racket and extends beyond the external component 1511 when juxtaposed, for easy removal of the external component 1511. In addition to hair clamp 1509, an additional hair clamp 1515 is utilized, which includes a magnet that is juxtaposed over, and attracted to and held in place by, the magnet associated with the external component 1511. Thus, hair 1505 may be clamped not only between hair clamp 1509 and the external component 1511, but also between hair clamp 1515 and the external component 1511, further contributing against gravitational/other forces to keep the external component 1511 properly positioned. In various embodiments, any magnet support associated with hair clamp 1515 does not need to radially extend as fully as the support associated with hair clamp 1509. In still further embodiments, hair clamp 1515 may be utilized without hair clamp 1509, with the external component 1511 juxtaposed directly over the skin/implantable component 1513.

In various embodiments, an attachment device may be used to attach the hair clamp loosely to the housing of the external component. This embodiment may be particularly advantageous if the hair clamp magnet is used with a small child to prevent swallowing. For example, the hair clamp and external component may be attached with a polyamide cord.

Rather than, or in addition to clamping hair, the hair clamp may be used to clamp other parts of the body. FIG. 16 shows, without limitation, an external component 1605 of a cochlear implant that, includes, for example, a magnet and coil that is juxtaposed and attracted to an implanted component (not shown) also having a magnet and coil, the coils for transcutaneous power and/or data transfer. Depending on the location of the implanted component, the external clamp may further be clamped to, without limitation, to the top of the outer ear 1601 or at the earlobe, via magnetic attraction to a hair (ear) clamp 1603.

For medical implants it may also be suitable to make use of other protruding (body) parts like, without limitation, the nose (or nose septum), nails and other positions. Of course not only body parts may be appropriate for clamping. The hair clamp/magnet may also apply for fixation of the external component/magnet 6 (with, for example, corresponding coil) to all proper items like clothes (e.g., hat, cap, sweatband, glasses).

In further embodiments of the invention, both or either the magnet support or the magnet of the hair clamp may include holes to allow for ventilation of the skin behind the hair clamp. Additionally, hair may be passed through/caught within the holes, providing increased force(s) against gravitation.

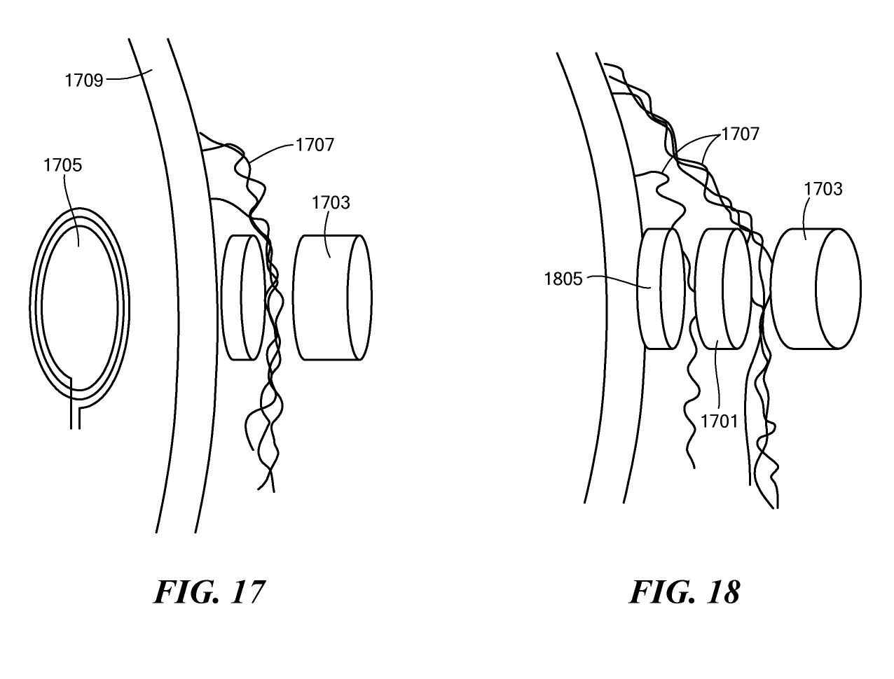

In various embodiments, the implantable component may not include a magnet. For example, the implantable component may include a coil for transcutaneous transmission of power and/or data, but no magnet. In such an embodiment, proper positioning of the external component/coil relative to the implanted magnet can be important for proper transmission of the power and/or data. In accordance with various embodiments of the invention, a hair clamp 1701 may be provided that is fixed to the skin 1709 of the user, as shown in FIG. 17. The hair clamp 1701 may be fixed to the skin of the user by, without limitation, skin glue. The hair clamp 1701 may include a first magnet and magnet support, as described in above embodiments. The hair clamp may advantageously serve as marker for correct positioning of the external component 1703 that provides, for example, optimum electrical coupling between the implanted coil 1705 and the coil of the external component 1703, without enforced magnetic attraction. Hair 1707 may be placed between the hair clamp 1701 and external component 1703. In addition to the magnetic force between the hair clamp 1701 and the external component 1703, the weight of the external component 1703 (that includes, for example, a housing, magnet, and primary coil) can also be held, to a large degree, by the friction force originating from hair clamp magnet and primary magnet against the patients hair.

Since the magnet of the hair clamp 1701 may be very strong, it could be torn off the skin or hair where it is fixed to (for e.g., by glue). To address this problem, a further hair clamp 1805 that includes a magnetisable or very weak magnet (or arrangement of magnets), resulting in a very low magnetic force, may be attached to the skin or hair, for example, by skin glue, as shown in FIG. 18, in accordance with an embodiment of the invention. The magnetic force of the hair clamp 1805 may be less than the adhesive force of the adhesive (e.g., the skin glue) to the skin.

Advantages of the above-described hair clamping system and methodology include, but are not limited to, the following:
The hair clamping technique and methodology is capable of automatically realigning internal and external magnets (and coils) for optimal coupling as result of the combined usage of magnetic and frictional force against drop off of the external component/coil.
Fixation technique of the external housing by summation of two forces (the magnetic and friction forces) for compensation of gravitational forces results in a stronger resulting force acting against drop off
The hair clamping technique and methodology results in advanced self-alignment and hence advanced coupling, especially for very thick skin flaps, since the hair clamp magnet will align much better than the external component as the ratio of (gravitational force)/(magnetically force) is outstanding.
The coil electromagnetic field (i.e. the field of data transmission) variation due to the hair clamp's magnet is negligible since, in typical embodiments, it adds to (or in certain embodiments can be considered a slice of) the external component's magnet.
The hair clamp may be designed with no gaps and a smooth surface, allowing for easy cleaning and increased hygienics.
In various embodiments, because of the increased attraction of the external component to the implanted component through use of the hair clamp magnet, smaller magnets may be used in the internal and external components compared to state-of-the-art devices, thus decreasing volume and/or weight.

The embodiments of the invention described above are intended to be merely exemplary; numerous variations and modifications will be apparent to those skilled in the art. All such variations and modifications are intended to be within the scope of the present invention.

What is claimed is:

1. A hair clamp for use with a partially implantable hearing device, the hair clamp comprising:
a first magnet for magnetically interacting with both an implanted magnet of the partially implantable hearing device, and a second magnet associated with an external component of the partially implantable hearing device; and
a magnet support coupled to the first magnet, the magnet support having a racket shape with a head portion and a handle portion extending therefrom, the first magnet coupled to the head portion.

2. The hair clamp according to claim 1, wherein the handle portion extends along a longitudinal axis from the head portion, and wherein the handle portion has an elevated portion along the longitudinal axis.

3. The hair clamp according to claim 2, wherein the elevated portion is elevated to substantially the height of the external component when the magnet of the external component is juxtaposed the first magnet.

4. The hair clamp according to claim 1, further comprising a plurality of magnets coupled to the head portion.

5. The hair clamp according to claim 1, wherein the head portion has a shape that is one of circular, rectangular, square, oval, elliptical and triangular.

6. The hair clamp according to claim 1, further comprising a coating of a material over the first magnet, the material providing increased friction relative to the first magnet.

7. The hair clamp according to claim 1, wherein the handle portion is removably attached to the head portion.

8. The hair clamp according to claim 1, wherein the first magnet is molded into the head portion.

9. A partially implantable hearing system, the system comprising:
a hair clamp including:
a first magnet; and
a magnet support coupled to the first magnet; and
an external component of the partially implantable hearing device, the external component including a housing having a second magnet; and
an implantable component of the partially implantable hearing device, the implantable component including a third magnet;
wherein when the second magnet is juxtaposed and aligned with the first magnet, the magnet support extends beyond the housing of the external component.

10. The partially implantable hearing system according to claim 9, wherein the magnet support has a racket shape with a head portion and a handle portion extending therefrom, the first magnet coupled to the head portion, the handle portion extending beyond the external component when the second magnet is juxtaposed and aligned with the first magnet.

11. The partially implantable hearing system according to claim 10, wherein the handle portion extends along a longitudinal axis from the head portion, and wherein the handle portion has an elevated portion along the longitudinal axis.

12. The partially implantable hearing system according to claim 11, wherein the elevated portion is elevated to substantially the height of the housing of the external component when the magnet of the external component is juxtaposed the first magnet.

13. The partially implantable hearing system according to claim 10, further comprising a plurality of magnets coupled to the magnet support.

14. The partially implantable hearing system according to claim 11, wherein the head portion has a shape that is one of circular, rectangular, square, oval, elliptical and triangular.

15. The partially implantable hearing system according to claim 10, wherein the external component includes a first coil, and the implantable component includes a second coil, the first coil and the second coil for transcutaneous transmission of at least one of a data signal and a power signal via electromagnetic coupling.

16. The partially implantable hearing system according to claim 10, wherein the partially implantable hearing system is a cochlear implant.

17. A method of positioning and removing an external component of a partially implantable hearing system relative to a user, respectively, the partially implantable hearing system including a hair clamp, the hair clamp including a first magnet held by a magnet support, the external component including a second magnet; the method comprising:
positioning the first magnet proximate a third magnet implanted in the user, such that hair clamping device is retained in a first position by magnetic forces between the first magnet and the third magnet;
positioning the second magnet proximate the first magnet, such that the external component is retained in a second position by magnetic forces between the second magnet and the first magnet, wherein the users hair is caught between the hair clamp and the external component, and wherein when the second magnet is juxtaposed and aligned with the first magnet in the second position, an extended portion of the magnet support extends beyond the housing of the external component.

18. The method of claim 17, further comprising:
holding down, by the user, on the extended portion of the magnet support; and
removing the external component from the second position, such that the external component is no longer retained in the second position by magnetic forces between the second magnet and the first magnet.

19. The method of claim 18, wherein removing the external component includes
sliding, by the user, the external component, until the magnetic fields of the second magnet is magnetically repelled away from the hair clamp.

20. The method of claim 17, wherein the magnet support has a racket shape with a head portion and a handle portion extending therefrom, the first magnet coupled to the head portion.
